(19) United States
(12) Patent Application Publication
(10) Pub. No.: US 2026/0257038 A1
(43) Pub. Date: Sep. 3, 2026

(54) PORTABLE INTERMITTENT URINARY CATHETER WITH LUBRICATION AND METHOD FOR USING SAME

(71) Applicant: GCMEDICA ENTERPRISE LTD. (WUXI), Wuxi (CN)

(72) Inventor: Sijiong Wu, Wuxi (CN)

(21) Appl. No.: 19/654,649

(22) Filed: Apr. 22, 2026

Related U.S. Application Data
(63) Continuation of application No. PCT/CN2024/099800, filed on Jun. 18, 2024.

(30) Foreign Application Priority Data
Oct. 30, 2023 (CN) 202311425688.3

(51) Int. Cl. A61M 25/01; A61M 25/00

(52) U.S. Cl. CPC A61M 25/01; A61M 25/002; A61M 25/007; A61M 2025/0175; A61M 2202/0014; A61M 2202/0496; A61M 2205/0222; A61M 2205/276; A61M 2209/06; A61M 2210/1089

(57) ABSTRACT

The present disclosure discloses a portable intermittent urinary catheter with lubrication and a method for using same. A rear end of a catheter of the urinary catheter is mounted on a telescopic assembly configured to drive the catheter to extend out of or retract into the telescopic assembly. The telescopic assembly is provided with drainage ports. When the catheter extends outside, urine can be discharged through the drainage ports. A telescopic assembly is detachably connected to a liquid storage assembly. A liquid storage chamber containing a lubricant is disposed in the liquid storage assembly. When the liquid storage chamber is in communication with the telescopic assembly, the lubricant in the liquid storage chamber is released, wets and lubricates the catheter. The method is convenient, employs a concealed catheter without a separately packaged lubricant, thereby offering portability, privacy protection, rapid lubrication, no-touch insertion, reduced infection risk, and resealability after use.

### FIELD OF THE INVENTION

The present disclosure relates to the technical field of medical instruments, and in particular, to a portable intermittent urinary catheter with lubrication and a method for using same.

### BACKGROUND

For patients with urinary incontinence, spinal cord injury, and neurogenic bladder, or those after urinary tract surgery, it is usually necessary to use intermittent urinary catheters to periodically empty their bladders, so as to reduce retention of urine and bladder pressure, prevent the occurrence of urinary tract infections and related complications, and promote recovery and healing.

Currently, a commonly used disposable intermittent urinary catheter consists of a catheter and a sheath, or includes only one catheter. The catheter is made of PVC or rubber materials, and is mostly sealed and packaged in a plastic packaging bag.

The existing disposable intermittent urinary catheter has some defects in use. For example, this packaging method occupies a large amount of space, and transparent packaging is not conducive to the protection of the user's personal privacy; the lubricant and the catheter are separately packaged, and a packaging bag containing the lubricant needs to be torn open before lubricating the catheter, which easily causes the lubricant to splash or be contaminated due to contact with the hand, resulting in a great change in operation; when the packaging bag is torn open or the catheter is directly exposed to the air and in contact with the hand during use, the risk of contamination is increased, which is not conducive to the user's health.

### SUMMARY OF THE INVENTION

To solve the above problems, the present disclosure provides a portable intermittent urinary catheter with lubrication and a method for using same. The catheter is provided in a concealed manner. The entire urinary catheter is portable and convenient to use, and effectively protects the privacy of users. A lubricant is directly contained in a liquid storage chamber, such that during use, the lubricant can be rapidly released to lubricate the catheter, and non-contact insertion of the catheter into a urethra is enabled, thereby effectively avoiding the exposure of the catheter and reducing the risk of contamination of the catheter. After use, the catheter can be resealed to reduce contamination.

According to one aspect of the present disclosure, a portable intermittent urinary catheter with lubrication is provided, which includes a catheter. The catheter is a tube body with a hollow structure, urine drainage holes are formed on a tube wall at a front end of the catheter, and the urinary catheter further includes:

a telescopic assembly, a rear end of the catheter being mounted on the telescopic assembly, the telescopic assembly being configured to drive the catheter to extend out of the telescopic assembly or retract into the telescopic assembly, the telescopic assembly being provided with drainage ports, wherein when the catheter extends out of the telescopic assembly, urine enters the catheter via the urine drainage holes and is discharged through the drainage ports; and

a liquid storage assembly, the liquid storage assembly being detachably connected to the telescopic assembly and covering the front end of the catheter, the liquid storage assembly being provided with a liquid storage chamber therein, the liquid storage chamber containing a lubricant, wherein when the liquid storage chamber is in communication with the telescopic assembly, the lubricant in the liquid storage chamber flows out, wets and lubricates the catheter.

Therefore, the telescopic assembly and the liquid storage assembly are assembled to form a chamber for accommodating the catheter. The rear end of the catheter is provided with the telescopic assembly for extension and retraction of the catheter. The liquid storage chamber is located at the front end of the catheter. When the urinary catheter is to be used, with the catheter in a retracted position, the liquid storage chamber is configured to be in communication with the telescopic assembly, and the lubricant contained in the liquid storage chamber is released to wet the catheter, such that the catheter is lubricated. After the catheter is lubricated, the telescopic assembly causes the catheter to extend gradually. The catheter is advanced toward the urethra bit by bit, that is, the catheter is screwed into the urethra bit by bit, rather than being entirely exposed to the air, which avoids the possibility of infection caused by inserting the entire catheter into the urethra while it is exposed to the air. After the catheter is inserted into the urethra, urine enters an interior of the catheter via the urine drainage holes and is discharged through the drainage ports. The intermittent urinary catheter with lubrication, by means of a housing formed by the telescopic assembly and the liquid storage assembly, conceals the catheter for urine drainage. The urinary catheter is portable and convenient to use, effectively protects the privacy of users, and does not require a separately packaged lubricant. The catheter can be lubricated in an enclosed space, making operation very convenient and avoiding exposure of the catheter. The telescopic assembly causes the catheter to extend, and then inserts the catheter into the urethra for use. The user's hand does not need to touch the catheter throughout the process, thereby enabling non-contact insertion into the urethra and reducing the risk of contamination of the catheter.

In some embodiments, the telescopic assembly includes a lower sleeve, a guide cylinder, and a sheath shaft; a rotary handle is disposed at a rear end of the guide cylinder, and the guide cylinder and the rotary handle form a step-like structure; the guide cylinder is a hollow tubular body, and a grille comprising a plurality of drainage ports is disposed in the guide cylinder; the lower sleeve is sleeved over the guide cylinder; a helical groove is formed on an inner wall of the lower sleeve; a Z-shaped sliding slot is formed on a cylinder body of the guide cylinder; a base having a stepped boss is disposed at the rear end of the catheter; the base is mounted to the sheath shaft, the sheath shaft is assembled in the Z-shaped sliding slot of the guide cylinder and is fitted into the helical groove of the lower sleeve, and when the rotary handle is rotated, the catheter extends or retracts along with the sheath shaft relative to the guide cylinder. Therefore, when the user holds the lower sleeve and rotates the rotary handle, the guide cylinder and the rotary handle rotate together, and the sheath shaft helically moves along the Z-shaped sliding slot of the guide cylinder and the helical groove of the lower sleeve such that the catheter extends outward. An end portion of the Z-shaped sliding slot of the guide cylinder acts as a stop to prevent the sheath shaft from extending excessively and slipping out. The retraction of the catheter is enabled by rotating the rotary handle in a reverse direction. After retraction, the catheter is accommodated in the guide cylinder.

In some embodiments, a guard plug is disposed in a center of the grille, the guard plug protrudes toward a side where the catheter is located and is embedded in the base of the catheter, and when the catheter extends along with the sheath shaft relative to the guide cylinder, the base is detached from the guard plug. Therefore, the guard plug, the guide cylinder, and the rotary handle are of an integrally formed structure, and the guard plug is tightly fitted to the base of the catheter to block the end of the catheter, such that the catheter can be positioned when the catheter is in a retraction state, the entry of impurities into the catheter from the drainage ports when the catheter is not in use can be avoided, and the lubrication of the catheter in a sealed state can also be facilitated.

In some embodiments, a hydrophilic coating is applied to a surface of the catheter, and a physiological saline solution is used as the lubricant. Therefore, a physiological saline solution is selected as the lubricant, and a hydrophilic coating is applied to the surface of the catheter in advance, such that when the physiological saline solution wets the catheter, the surface of the catheter attracts water molecules, thereby achieving a better lubricating effect and reducing the discomfort during the insertion of the catheter into the urethra.

In some embodiments, the liquid storage assembly includes a first upper sleeve and a first rotary cap. Both ends of the first upper sleeve are connected to the lower sleeve and the first rotary cap, respectively. A sealing sheet is disposed on a side, close to the lower sleeve, of the first upper sleeve. The first rotary cap, the first upper sleeve, and the sealing sheet form the liquid storage chamber. The first rotary cap is provided with a cutter therein. After the cutter breaks the sealing sheet, the lubricant in the liquid storage chamber enters the guide cylinder, wets and lubricates the catheter. Therefore, the lubricant is contained in the first upper sleeve, the first rotary cap is assembled to the first upper sleeve, the cutter is immersed in the lubricant, and then the first upper sleeve of the liquid storage assembly is assembled to the lower sleeve. As a result, the hermetically stored lubricant may not leak. When there is a need to lubricate the catheter, the cutter is used to break the sealing sheet, such that the lubricant flows out from the broken sealing sheet and directly enters the guide cylinder, wets and lubricates the catheter.

In some embodiments, a side, facing the first rotary cap, of the sealing sheet is provided with a flange perpendicular to the sealing sheet; the cutter includes a curved surface, a protruding rib, and a tip; the curved surface and the protruding rib are connected to a side of the first rotary cap, a vertical surface of the protruding rib is connected to the curved surface, and the tip is disposed at an end portion of the curved surface; by rotating the first rotary cap, the tip presses against and cuts the flange of the sealing sheet to break the sealing sheet. Therefore, when the first rotary cap is rotated, the tip of the cutter pushes the flange on the sealing sheet, generating a pressing-and-cutting effect by means of the pushing force to break the sealing sheet and allowing the lubricant to be released into the guide cylinder.

In some embodiments, a side, close to the lower sleeve, of the first upper sleeve is provided with a first outer wall and a first inner wall; the first outer wall is longer than the first inner wall; and the first outer wall is threadedly connected to the lower sleeve, and the first inner wall is disposed in the lower sleeve and abuts against the guide cylinder; the sealing sheet is disposed on the first inner wall of the first upper sleeve; a sealing ring is disposed at a junction of the first rotary cap and the first upper sleeve. Therefore, the sealing ring can ensure a better sealing effect of the formed liquid storage chamber. The design of the inner and outer walls of the first upper sleeve not only facilitates the mounting of the first upper sleeve and the lower sleeve, but is also beneficial to allowing the lubricant to accurately be released into the guide cylinder upon breaking the sealing sheet to prevent the lubricant from leaking onto the entire outer wall of the urinary catheter, and helping maintain hand hygiene.

In some embodiments, a first tamper-evident ring is disposed at an end, close to the lower sleeve, of the first upper sleeve; a plurality of clips are disposed in the first tamper-evident ring; by unscrewing the first upper sleeve, the clips are pressed by threads, such that the first tamper-evident ring is broken. Therefore, the first tamper-evident ring is provided to indicate that the catheter is a disposable product, thereby helping improve safety.

In some embodiments, the liquid storage assembly includes a second upper sleeve and a second rotary cap, and both ends of the second upper sleeve are connected to the lower sleeve and the second rotary cap, respectively; a side, close to the lower sleeve, of the second upper sleeve is provided with a second outer wall and a second inner wall; the second outer wall is longer than the second inner wall; and the second outer wall is threadedly connected to the lower sleeve, and the second inner wall is disposed in the lower sleeve and abuts against the guide cylinder; a protruding disc is connected to the second inner wall, a cavity opening is formed on the second inner wall, and a bottom of the cavity opening extends above the guide cylinder; an arc-shaped sliding slot is formed on a side, close to the second rotary cap, of the second upper sleeve, and the second rotary cap is provided with a sheath rod matched with the arc-shaped sliding slot; a top end of the second rotary cap abuts against the protruding disc, such that the liquid storage chamber is formed between an interior of the second rotary cap and the protruding disc; when the second rotary cap is rotated to be separated from the protruding disc, the lubricant enters the guide cylinder via the cavity opening, wets and lubricates the catheter; an end, close to the lower sleeve, of the second upper sleeve is provided with a second tamper-evident ring. Therefore, the lubricant is contained in the liquid storage chamber formed by assembling the second rotary cap to the first upper sleeve. When there is a need to lubricate the catheter, the second rotary cap is rotated relative to the first upper sleeve, the sheath rod is guided along the arc-shaped sliding slot, a lower end of the second rotary cap is separated from the protruding disc of the first upper sleeve, and the lubricant flows into the guide cylinder from the cavity opening, wets and lubricates the catheter.

According to another aspect of the present disclosure, a method for using the portable intermittent urinary catheter with lubrication described above is provided, which includes steps of:

S1a, rotating the first rotary cap, such that the first rotary cap is misaligned with the first upper sleeve, and a tip of the first rotary cap presses and cuts the flange of the sealing sheet to break the sealing sheet, and the lubricant in the liquid storage chamber enters the guide cylinder, wets and lubricates the catheter; and rotating the first upper sleeve, the clips of the first tamper-evident ring are pressed by the threads, such that the first tamper-evident ring is broken;

S2a, continuing to rotate the first upper sleeve, such that the first upper sleeve is separated from the lower sleeve;

S3a, rotating the rotary handle, such that the catheter extends along with the sheath shaft relative to the guide cylinder, the base is detached from the guard plug, and an interior of the catheter is in communication with the drainage ports;

S4a, allowing the catheter to gradually extend, and gradually screwing the catheter into the urethra of the user, such that urine enters the interior of the catheter via the urine drainage holes and is discharged through the drainage ports, and the drained urine may be directly directed into a toilet or into a urine collection bag via an intervening connector; and

S5a, upon completion of urine drainage, rotating the rotary handle in a reverse direction, such that the catheter retracts along with the sheath shaft relative to the guide cylinder, the guard plug is embedded in the base of the catheter, and the first upper sleeve assembled with the first rotary cap is sealed to the lower sleeve by threaded connection.

According to another aspect of the present disclosure, a method for using the portable intermittent urinary catheter with lubrication described above is provided, which includes steps of:

S1b, rotating the second rotary cap, such that the second rotary cap is separated from the protruding disc, and the lubricant in the liquid storage chamber enters the guide cylinder via the cavity opening, wets and lubricates the catheter;

S2b, rotating the second upper sleeve, such that the second upper sleeve is separated from the lower sleeve, and as the second upper sleeve rotates, the second tamper-evident ring is caused to break;

S3b, rotating the rotary handle, such that the catheter extends along with the sheath shaft relative to the guide cylinder, the base is detached from the guard plug, and an interior of the catheter is in communication with the drainage ports;

S4b, allowing the catheter to gradually extend and be screwed into the urethra of the user, such that urine enters the interior of the catheter via the urine drainage holes and is discharged through the drainage ports, and the drained urine may be directly directed into a toilet or into a urine collection bag via an intervening connector; and

S5b, upon completion of urine drainage, rotating the rotary handle in a reverse direction, such that the catheter retracts along with the sheath shaft relative to the guide cylinder, the guard plug is embedded in the base of the catheter, the second upper sleeve assembled with the second rotary cap is sealed to the lower sleeve by threaded connection, and the second rotary cap is rotated back to be reset relative to the second upper sleeve.

Compared with the prior art, the present disclosure has the following beneficial effects:
(1) The portable intermittent urinary catheter with lubrication accommodates the catheter therein in a concealed manner. The overall design is compact and is convenient for the user to carry and does not readily expose the user's privacy.
(2) The lubricant is directly pre-stored in the liquid storage chamber above the catheter, without the need for a separately packaged lubricant. Simply by rotating the first rotary cap or the second rotary cap, the lubricant can be released, such that the catheter can be lubricated quickly, thereby avoiding the splashing of the lubricant when the packaging of the lubricant is torn open, and also reducing the possibility of the catheter being exposed to the air and contaminated from the outside after the packaging bag is torn open. During the lubrication of the catheter, the user's hand does not come into contact with the catheter.
(3) The catheter is provided with the telescopic assembly, and by rotating the rotary handle, the extension or retraction of the catheter is enabled. During the insertion of the catheter into the urethra, the catheter is advanced toward the urethra bit by bit; that is, the catheter is screwed into the urethra bit by bit, rather than being entirely exposed to the air. In this way, the possibility of infection caused by inserting the entire catheter into the urethra while it is exposed to the air is avoided, and non-contact insertion is enabled throughout the process.
(4) After use, the catheter is retracted for accommodation, such that the catheter can be resealed and discarded, thereby preventing the generated waste from causing contamination to the environment.

### BRIEF DESCRIPTION OF THE DRAWINGS

FIG. 1 is a perspective view of a portable intermittent urinary catheter with lubrication according to one embodiment of the present disclosure;

FIG. 2 is a schematic view of an internal structure of a portable intermittent urinary catheter with lubrication according to one embodiment;

FIG. 3 is a schematic perspective view of a first rotary cap;

FIG. 4 is a schematic structural view of a first rotary cap and a sealing sheet, where FIG. 4A is a schematic view of the first rotary cap and the sealing sheet from one perspective when the sealing sheet is in a sectional view, and FIG. 4B is a schematic view of the first rotary cap and the sealing sheet from another perspective when the sealing sheet is intact;

FIG. 5 is a perspective view of a first upper sleeve;

FIG. 6 is a schematic structural view of a first upper sleeve, where FIG. 6A is a front view of the first upper sleeve, and FIG. 6B is a schematic view of an internal structure of the first upper sleeve;

FIG. 7 is a schematic structural view of a lower sleeve, where FIG. 7A is a perspective view of the lower sleeve, and FIG. 7B is a schematic view of an internal structure of the lower sleeve;

FIG. 8 is a perspective view of a guide cylinder;

FIG. 9 is a schematic view of FIG. 1 from another perspective;

FIG. 10 is a perspective view of a portable intermittent urinary catheter with lubrication according to another embodiment of the present disclosure;

FIG. 11 is a schematic view of FIG. 10 from another perspective;

FIG. 12 is a schematic structural view of a second rotary cap, where FIG. 12A is a perspective view of the second rotary cap, and FIG. 12B is a schematic structural view of the second rotary cap without a lid assembled; and

FIG. 13 is a schematic structural view of a second upper sleeve, where FIG. 13A is a perspective view of the second upper sleeve, and FIG. 13B is a schematic view of an internal structure of the second upper sleeve.

### DETAILED DESCRIPTION OF THE PREFERRED EMBODIMENTS

The present disclosure is further described below with reference to specific embodiments.

Embodiment 1

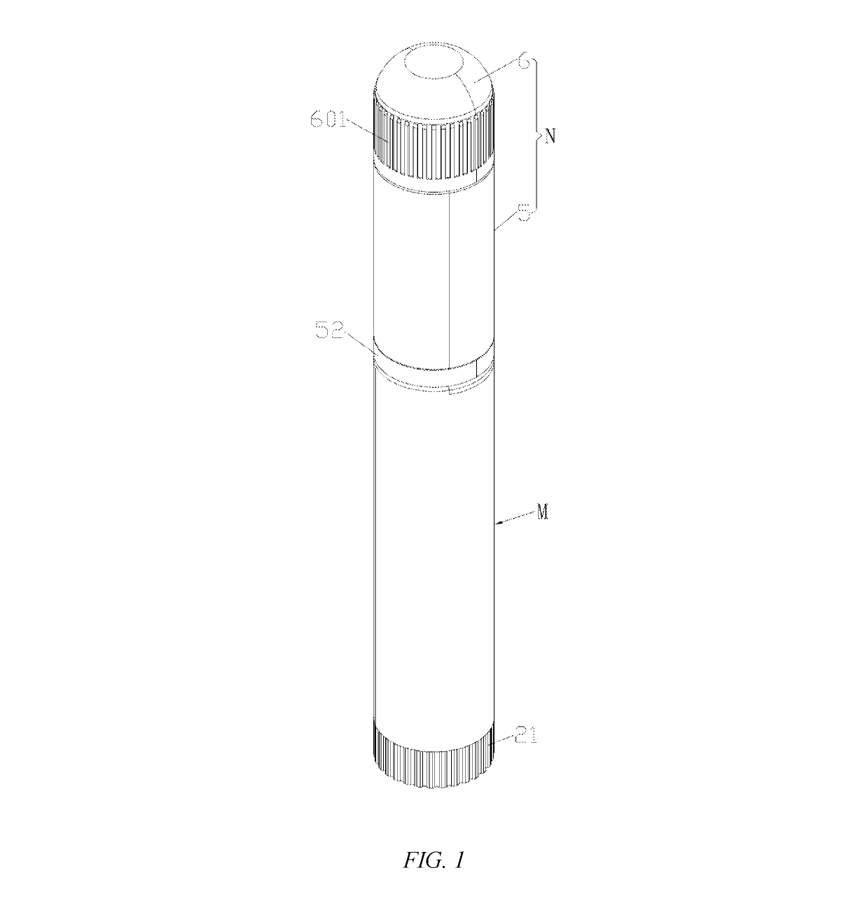

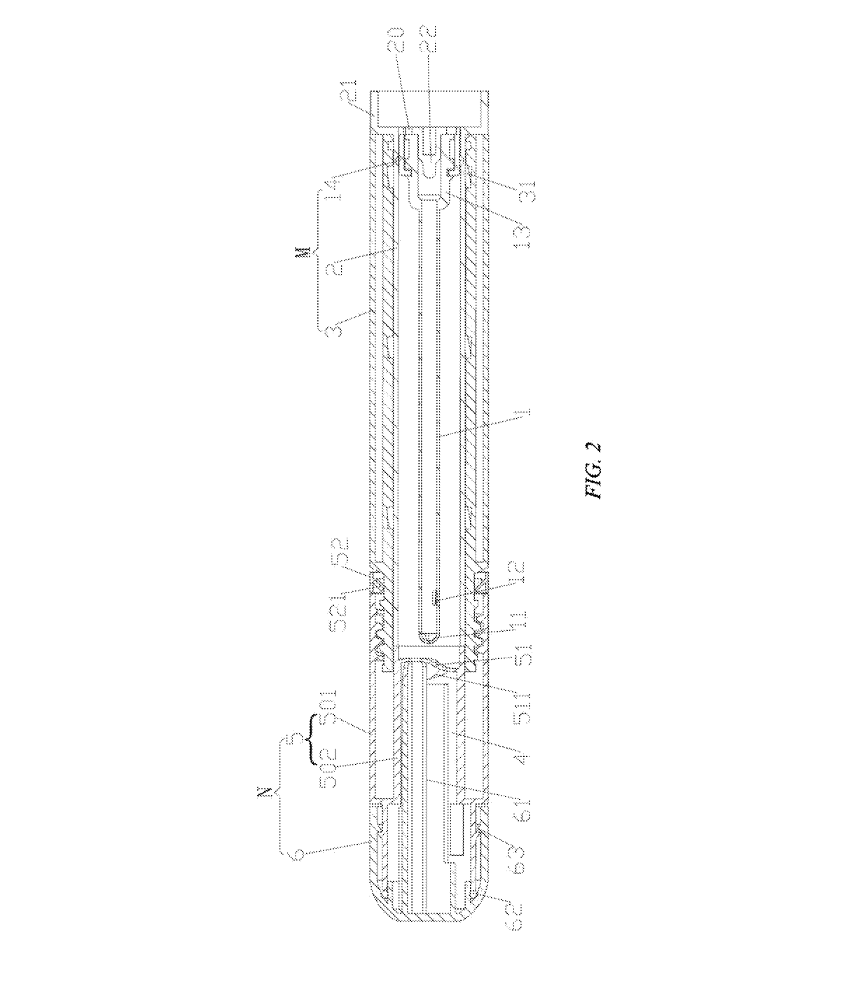

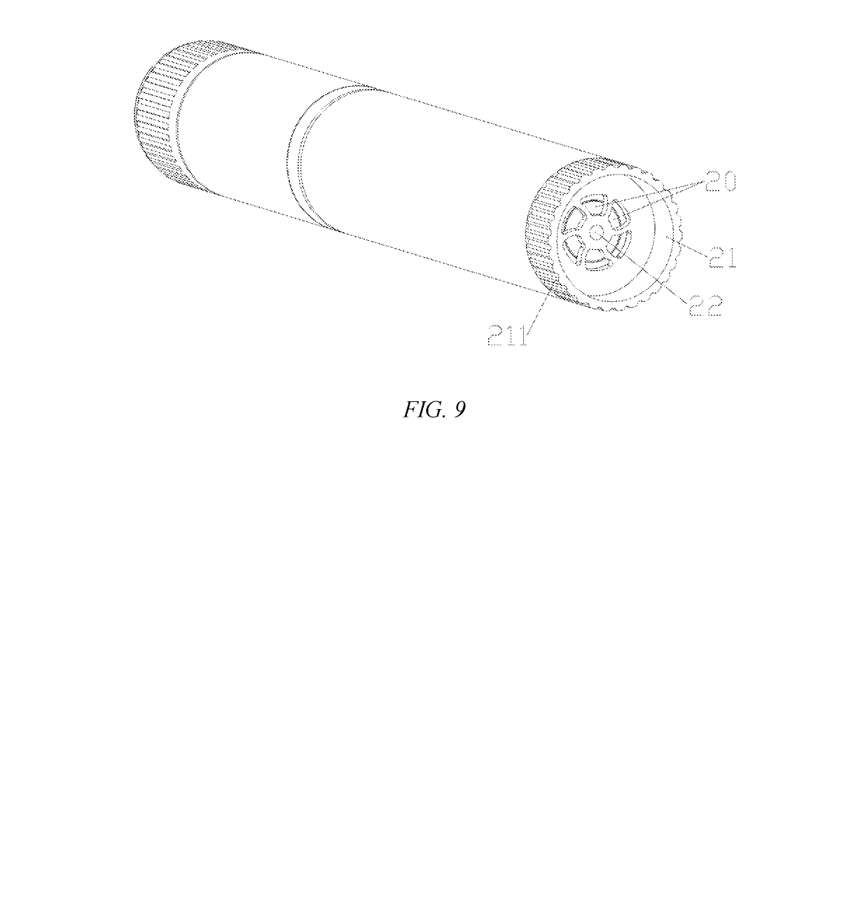

As shown in FIGS. 1 and 2, a portable intermittent urinary catheter with lubrication according to one embodiment of the present disclosure includes a catheter 1. The catheter 1 is a tube body with a hollow structure, and a front end portion of the catheter 1 is an arc-shaped tip 11, such that a user can smoothly insert the catheter into the urethra. A pair of urine drainage holes 12 are formed on two sides of a tube wall, close to a front end, of the catheter 1 by using a laser drilling technology. The urine drainage holes 12 are elliptical polished holes. In practical applications, the urine drainage holes 12 may also be of other shapes, such as circular. The urinary catheter further includes a telescopic assembly M and a liquid storage assembly N. A rear end of the catheter 1 is mounted on the telescopic assembly M. The telescopic assembly M can drive the catheter 1 to extend out of the telescopic assembly M or retract into the telescopic assembly M. The telescopic assembly M is provided with drainage ports 20 (shown in FIG. 9). When the catheter 1 extends out of the telescopic assembly M, the catheter 1 can be in communication with the drainage ports 20, and urine can enter the catheter 1 via the urine drainage holes 12 and be drained from the drainage ports 20. The liquid storage assembly N is detachably connected to the telescopic assembly M, and when the liquid storage assembly N and the telescopic assembly M are assembled together, a cavity for accommodating the catheter 1 is formed and covers the catheter 1. The liquid storage assembly N is provided with a liquid storage chamber 4 therein, the liquid storage chamber containing a lubricant. When the liquid storage chamber 4 is in communication with the telescopic assembly M, the lubricant in the liquid storage chamber 4 flows out, wets and lubricates the catheter 1.

The telescopic assembly M and the liquid storage assembly N are assembled to form a chamber for accommodating the catheter 1. The rear end of the catheter 1 is provided with the telescopic assembly M that allows the catheter 1 to extend and retract. The liquid storage chamber 4 is located at the front end of the catheter 1. When there is a need to use the urinary catheter and the catheter 1 is in a retracted position, the liquid storage chamber 4 is in communication with the telescopic assembly M, and the lubricant stored in the liquid storage chamber 4 flows down to wet the catheter 1, such that the catheter 1 is lubricated. After the catheter 1 is lubricated, the telescopic assembly M causes the catheter 1 to extend, so as to insert the catheter 1 into the urethra. The urine enters the interior of the catheter 1 via the urine drainage holes 12 and is then drained via the drainage ports 20.

The intermittent urinary catheter with lubrication, by means of a housing formed by the telescopic assembly M and the liquid storage assembly N, conceals the catheter 1 for urine drainage. The catheter is portable and convenient to use, effectively protects the privacy of the user, and does not require an additional lubricant that is separately packaged. The catheter 1 can be lubricated in an enclosed space, making operation very convenient and avoiding exposure of the catheter 1. The telescopic assembly M causes the catheter 1 to extend, and then gradually rotates and inserts the catheter into the urethra for use. The user's hand does not need to touch the catheter 1 throughout the process, thereby enabling non-contact insertion into the urethra and reducing the risk of contamination of the catheter 1. During the insertion of the catheter 1 into the urethra, the catheter is advanced toward the urethra bit by bit; that is, the catheter 1 is screwed into the urethra bit by bit, rather than being fully extended at once, which would expose the entire catheter 1 to the air. In this way, the possibility of infection caused by inserting the entire catheter 1 into the urethra while it is exposed to the air is avoided.

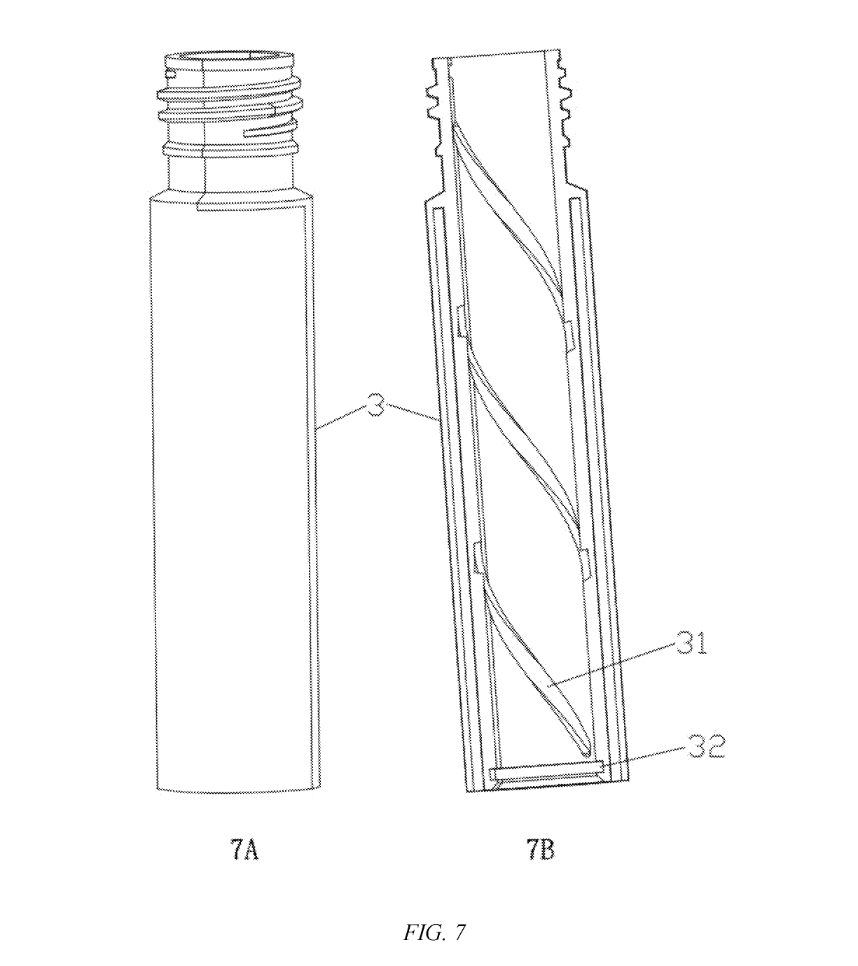

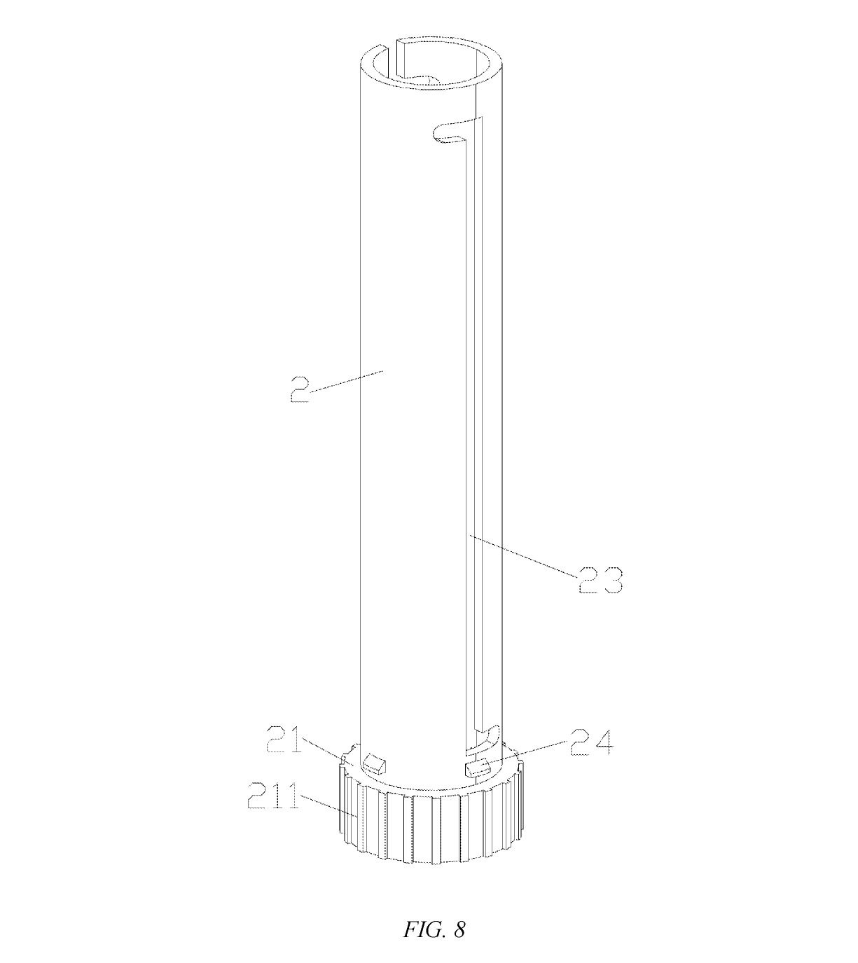

Specifically, as shown in FIG. 2 and FIGS. 7 to 9, the telescopic assembly M includes a lower sleeve 3, a guide cylinder 2, and a sheath shaft 14. The guide cylinder 2 is a hollow tubular body, a rotary handle 21 is disposed at a rear end of the guide cylinder 2 in an extending manner, a ring of anti-slip ribs 211 is formed on an outer wall of the rotary handle 21, and the guide cylinder 2 and the rotary handle 21 form a step-like structure. At a connection position between the guide cylinder 2 and the rotary handle 21, a grille comprising a plurality of drainage ports 20 is disposed in the guide cylinder 2, and the plurality of drainage ports 20 are of a sector-shaped hollow structure. A guard plug 22 is disposed in the center of the grille, and the guard plug 22 protrudes toward a side where the catheter 1 is located. A base 13 of a stepped boss is disposed at the rear end of the catheter 1 in an extending manner. The base 13 is also hollow, and the interior of the base is in communication with the catheter 1. The guard plug 22 can be embedded in the base 13 of the catheter 1. When the guard plug 22 is embedded in the base 13, the drainage ports 20 are blocked. The guard plug 22 is step-like and a top end of the guard plug is a circular arc-shaped end, thereby facilitating the assembly of the base 13 of the catheter 1 and the guard plug 22, and allowing the catheter 1 to dock with and insert into the guard plug 22 when the catheter returns to its original position. The guard plug 22, the guide cylinder 2, and the rotary handle 21 are of an integrally formed structure, and the guard plug 22 is tightly fitted to the base 13 of the catheter 1 to block the end of the catheter 1, such that the catheter 1 can be positioned when the catheter 1 is in a retracted state, the entry of impurities into the catheter 1 from the drainage ports 20 when the catheter 1 is not in use can be avoided, and the lubrication of the catheter 1 in a sealed state can also be facilitated. The lower sleeve 3 is sleeved over and assembled outside the guide cylinder 2 and is engaged with a track groove 32 of the lower sleeve 3 by means of several clips 24 at a root part of the guide cylinder 2, thereby preventing the guide cylinder 2 from disengaging from the lower sleeve 3, and allowing the guide cylinder 2 to rotate within the lower sleeve 3. A continuous helical groove 31 is formed on the inner wall of the lower sleeve 3. To better provide the helical groove 31 and maintain a sealed space for the lubricant to lubricate the catheter 1, a part of the lower sleeve 3 that is not connected to the liquid storage assembly N is designed with double-layer inner and outer walls. The helical groove 31 is formed inside the inner wall, with the groove bottom not penetrating through the inner wall itself. A Z-shaped sliding slot 23 is formed on a cylinder body of the guide cylinder 2. The sheath shaft 14 is mounted outside the base 13, and the sheath shaft 14 is assembled in the Z-shaped sliding slot 23 of the guide cylinder 2 and is fitted into the helical groove 31 of the lower sleeve 3. When the rotary handle 21 is rotated, the catheter 1 undergoes an extension motion or a retraction motion along with the sheath shaft 14 relative to the guide cylinder 2. An end portion of the Z-shaped sliding slot 23 of the guide cylinder 2 forms a stop, thereby preventing the sheath shaft 14 from protruding excessively and slipping out.

When the user holds the lower sleeve 3 and rotates the rotary handle 21, the guide cylinder 2 and the rotary handle 21 rotate together, and the sheath shaft 14 helically ascends along the Z-shaped sliding slot 23 of the guide cylinder 2 and the helical groove 31 of the lower sleeve 3, thereby causing the catheter 1 to extend. When the catheter 1 undergoes an extension motion along with the sheath shaft 14 relative to the guide cylinder 2, the base 13 is detached from the guard plug 22, thereby enabling the interior of the catheter 1 to be in communication with the drainage ports 20. When the rotary handle 21 is rotated in a reverse direction, the retraction of the catheter 1 is enabled. After retraction, the catheter 1 is accommodated in the guide cylinder 2, and finally the base 13 of the catheter 1 is inserted back into the guard plug 22 at the rotary handle 21.

During the manufacturing of the catheter 1, a hydrophilic coating is applied to the surface of the catheter 1. A physiological saline solution is selected as the lubricant. A hydrophilic coating is applied to the surface of the catheter 1 in advance, such that when the physiological saline solution wets the catheter 1, the surface of the catheter 1 attracts water molecules, thereby achieving a better lubricating effect and reducing the discomfort during the insertion of the catheter 1 into the urethra.

In this embodiment, as shown in FIG. 2, the liquid storage assembly N includes the first upper sleeve 5 and the first rotary cap 6. Both ends of the first upper sleeve 5 are connected to the lower sleeve 3 and the first rotary cap 6, respectively. A sealing sheet 51 is disposed on a side, close to the lower sleeve 3, of the first upper sleeve 5. The first rotary cap 6, the first upper sleeve 5, and the sealing sheet 51 form the liquid storage chamber 4. The first rotary cap 6 is provided with a cutter 61 therein. After the cutter 61 breaks the sealing sheet 51, the lubricant in the liquid storage chamber 4 enters the guide cylinder 2, wets and lubricates the catheter 1. An outer wall of the first rotary cap 6 is provided with anti-slip ribs 601 (shown in FIG. 1).

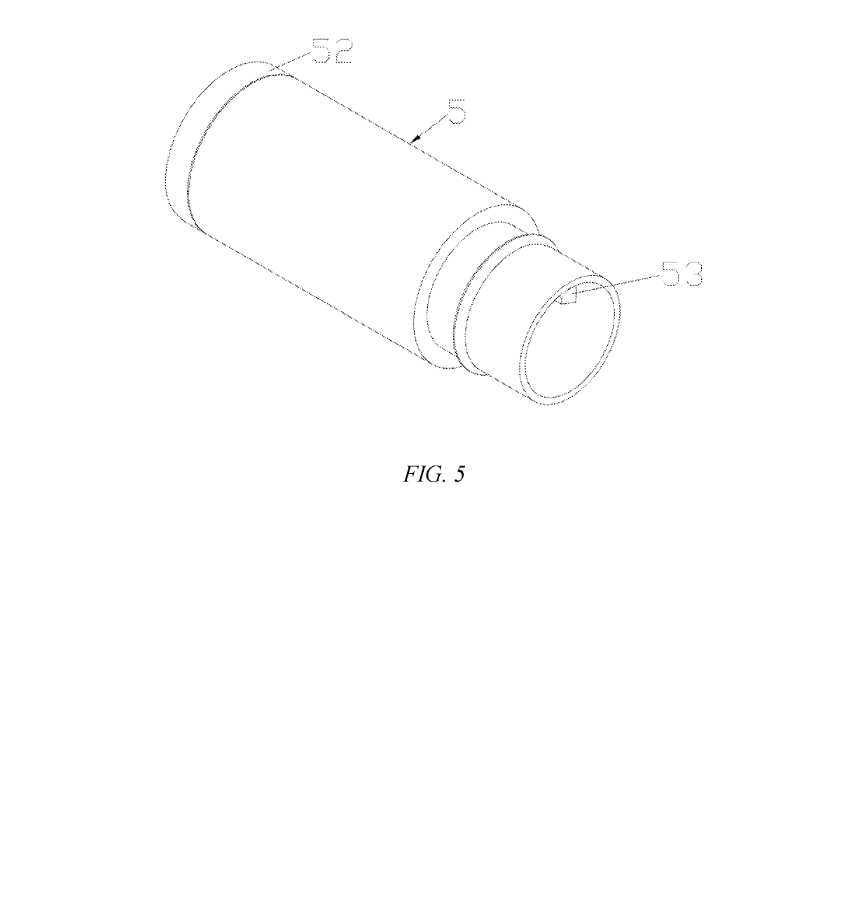

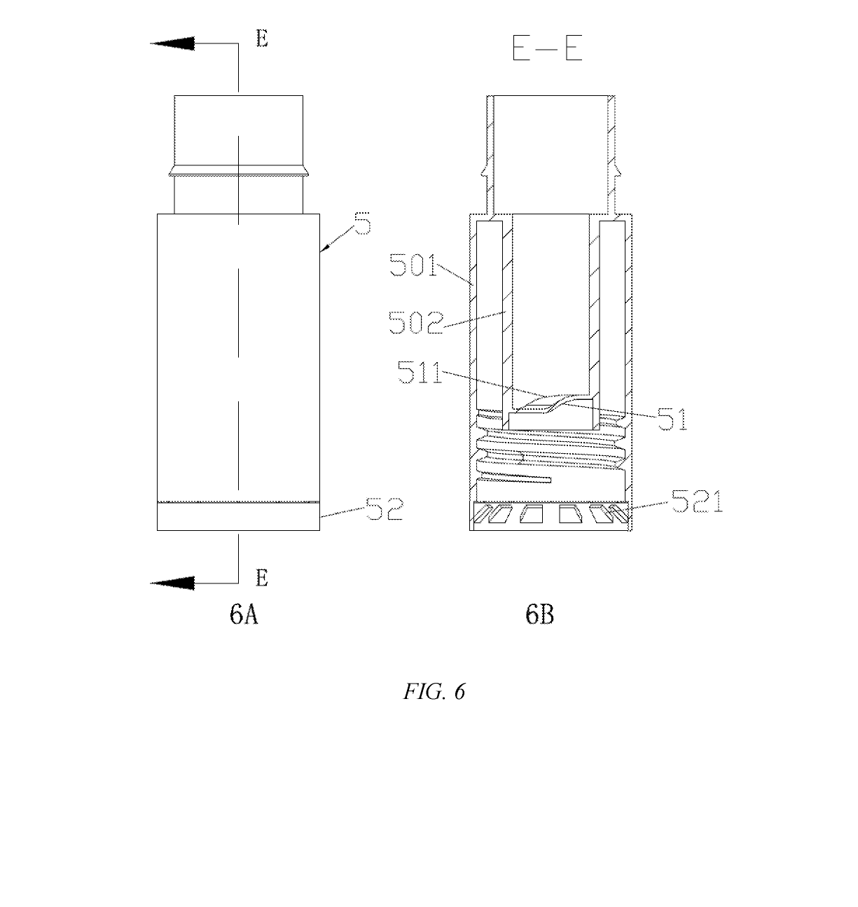

Specifically, as shown in FIGS. 5 and 6, a side, close to the lower sleeve 3, of the first upper sleeve 5 is provided with a first outer wall 501 and a first inner wall 502. The first outer wall 501 is longer than the first inner wall 502. In addition, the first outer wall 501 is threadedly connected to the lower sleeve 3, and the first inner wall 502 is disposed in the lower sleeve 3 and abuts against the guide cylinder 2. The sealing sheet 51 is disposed on the first inner wall 502 of the first upper sleeve 5. The sealing sheet 51 is integrally injection-molded with the first upper sleeve 5, and the wall thickness of the sealing sheet 51 is thinner than that of the first inner wall 502. During assembly, a sealing ring 62 is mounted at the junction of the first rotary cap 6 and the first upper sleeve 5, the sealing ring 62 can ensure a better sealing effect of the formed liquid storage chamber 4, and the first rotary cap 6 is snap-fitted to the first upper sleeve 5 by means of clips 63. The design of the inner and outer walls of the first upper sleeve 5 not only facilitates the mounting of the first upper sleeve 5 and the lower sleeve 3, but is also beneficial to allowing the lubricant to accurately flow into the guide cylinder 2 upon breaking the sealing sheet 51, thereby preventing the lubricant from leaking onto the outer wall of the entire urinary catheter, and helping maintain hand hygiene.

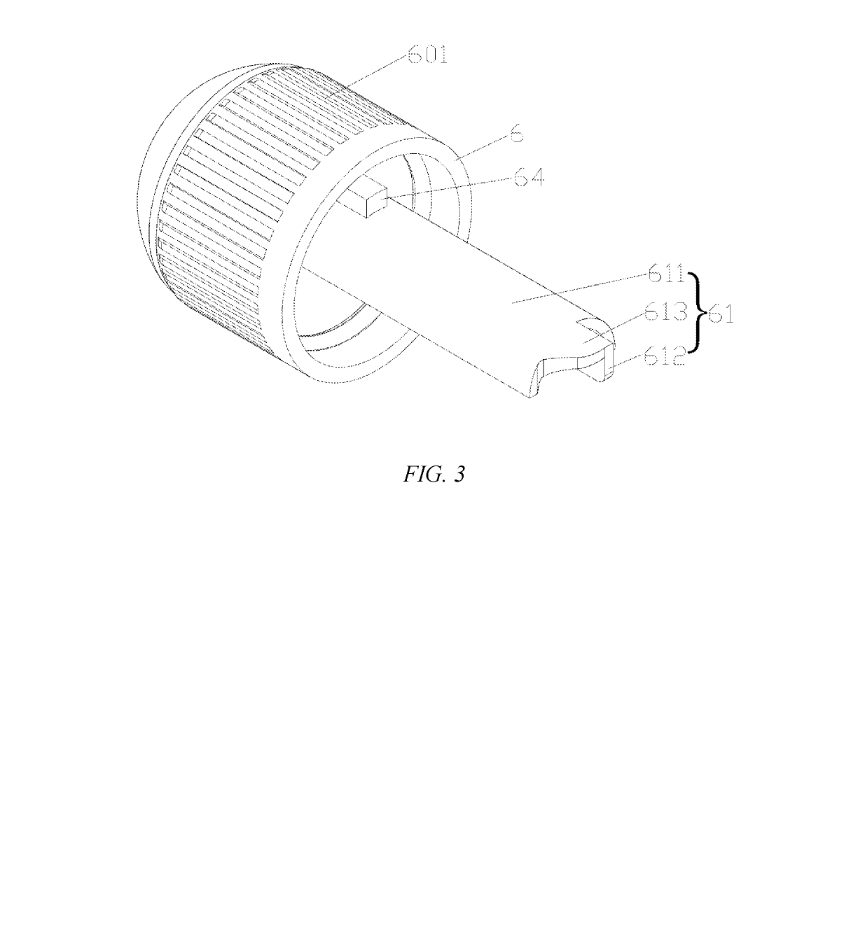

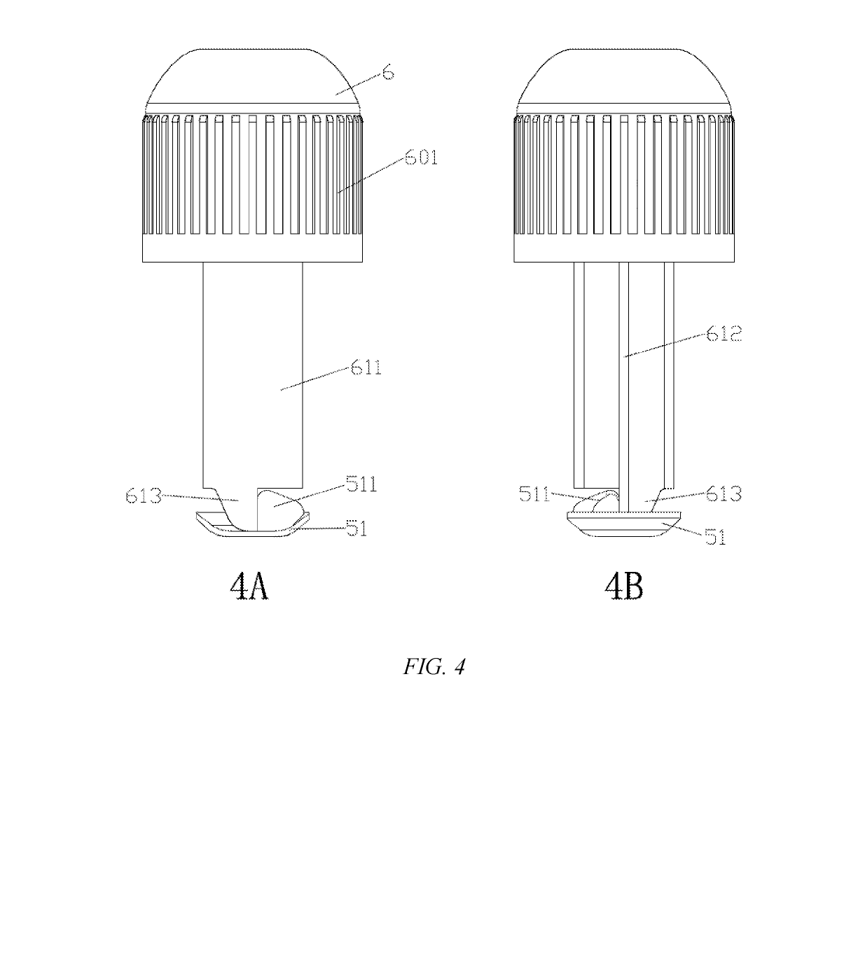

A side, facing the first rotary cap 6, of the sealing sheet 51 is provided with a flange 511 perpendicular to the sealing sheet 51, and the flange 511 and the sealing sheet 51 are integrally formed. As shown in FIGS. 3 and 4, the cutter 61 includes a curved surface 611, a protruding rib 612, and a tip 613. The curved surface 611 and the protruding rib are connected to a side of the first rotary cap 6, a vertical surface of the protruding rib is connected to the curved surface 611, and the tip 613 is disposed at an end portion of the curved surface 611. By rotating the first rotary cap 6, the tip 613 presses against and cuts the flange 511 of the sealing sheet 51 to break the sealing sheet 51.

To indicate that the urinary catheter is a disposable product and improve safety, as shown in FIG. 2, a first tamper-evident ring 52 is disposed at an end, close to the lower sleeve 3, of the first upper sleeve 5. The first tamper-evident ring 52 is at a bottom end of the first outer wall 501 of the first upper sleeve 5, and a plurality of clips 521 are disposed in the first tamper-evident ring 52. The first rotary cap 6 is provided with a vertical rotary cap protruding rib 64 therein (shown in FIG. 3), and an inner wall of the interior of an upper end of the first upper sleeve 5 is provided with a vertical upper sleeve protruding rib 53 (shown in FIG. 5). When the first rotary cap 6 is rotated, the rotary cap protruding rib 64 misalignedly pushes the upper sleeve protruding rib 53 to rotate apart, and as the first upper sleeve 5 ascends relative to the lower sleeve 3, the clips are pressed by the threads, such that the first tamper-evident ring 52 is broken. When the first rotary cap 6 is rotated, the tip 613 of the cutter 61 pushes the flange 511 on the sealing sheet 51, generating a pressing-and-cutting effect by means of the pushing force, to break the sealing sheet 51 and allowing the lubricant to flow into the guide cylinder 2.

The lubricant is contained in the first upper sleeve 5, the first rotary cap 6 is assembled to the first upper sleeve 5, the cutter 61 is immersed in the lubricant, and then the first upper sleeve 5 of the liquid storage assembly N is assembled to the lower sleeve 3. As a result, the lubricant stored in a sealed manner may not leak. When there is a need to lubricate the catheter 1, the cutter 61 is used to break the sealing sheet 51, such that the lubricant flows out from the broken sealing sheet 51 and directly enters the guide cylinder 2, wets and lubricates the catheter 1.

A method for using the portable intermittent urinary catheter with lubrication described above includes the following steps:

In S1a, the first rotary cap 6 is rotated, such that the rotary cap protruding rib 64 pushes the upper sleeve protruding rib 53, then the first upper sleeve 5 rotates, the first upper sleeve 5 ascends relative to the lower sleeve 3, and the clips 521 of the first tamper-evident ring 52 are pressed by threads, such that the first tamper-evident ring 52 is broken; the first rotary cap 6 is misaligned with the first upper sleeve 5, and the rotary cap 6 is rotated, such that the tip 613 of the first rotary cap 6 presses and cuts the flange 511 of the sealing sheet 51 to break the sealing sheet 51, and the lubricant in the liquid storage chamber 4 enters the guide cylinder 2, wets and lubricates the catheter 1.

In S2a, the first upper sleeve 5 is rotated, such that the first upper sleeve 5 is separated from the lower sleeve 3.

In S3a, the liquid storage assembly N composed of the first rotary cap 6 and the first upper sleeve 5 is set aside. The telescopic assembly M is operated, and the rotary handle 21 is rotated, such that the catheter 1 undergoes an extension motion along with the sheath shaft 14 relative to the guide cylinder 2, the base 13 is detached from the guard plug 22, and the interior of the catheter 1 is in communication with the drainage ports 20. Specifically, a channel for fluid drainage is formed in the interior of the catheter 1, the interior of the guide cylinder 2, and the drainage ports 20.

In S4a, a head portion of the catheter 1 extends and is inserted into the urethra of the user. Then, as the catheter 1 gradually extends, the catheter 1 is gradually screwed into the urethra of the user bit by bit, and urine enters the interior of the catheter 1 via the urine drainage holes 12 and is drained via the drainage ports 20. The drained urine may be directed directly into a toilet or be connected to a urine collection bag via an intervening connector.

In S5a, upon completion of urine drainage, the rotary handle 21 is rotated in a reverse direction, such that the catheter 1 undergoes a retraction motion along with the sheath shaft 14 relative to the guide cylinder 2, the guard plug 22 is embedded in the base 13 of the catheter 1, and the first upper sleeve 5 assembled with the first rotary cap 6 is sealed to the lower sleeve 3 by threaded connection. Finally, the used urinary catheter may be disposed of as medical waste.

Embodiment 2

Embodiment 2 differs from Embodiment 1 only in that the structure of the liquid storage assembly N is different.

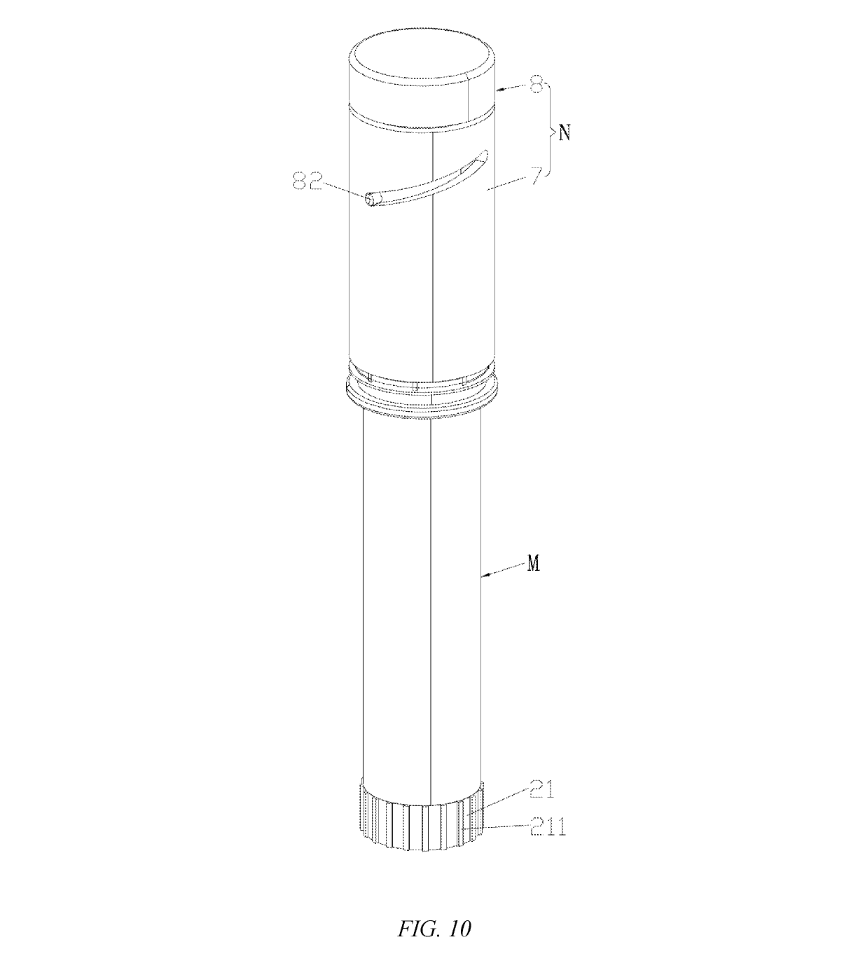

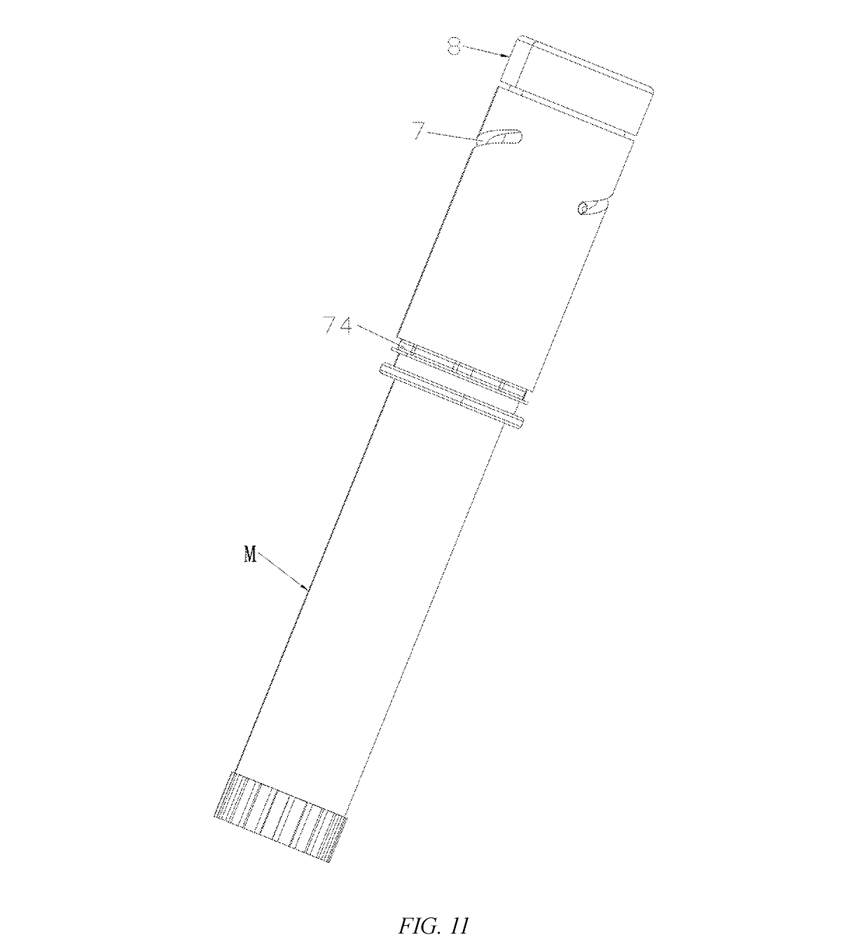

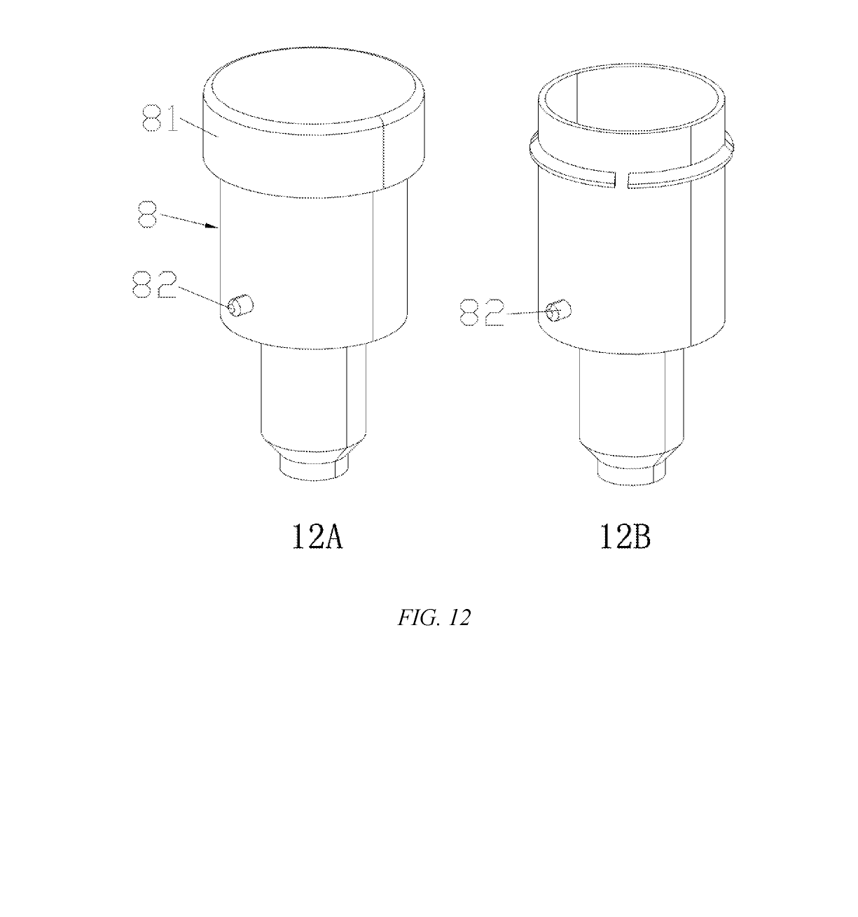

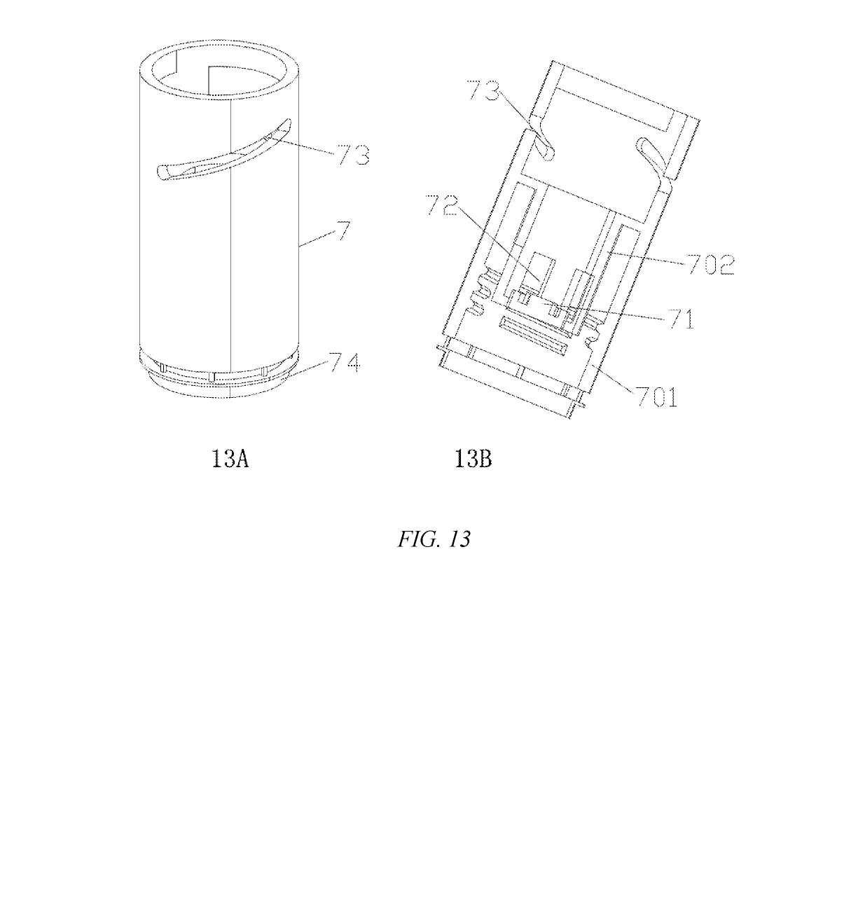

Specifically, as shown in FIGS. 10 to 13, the liquid storage assembly N includes a second upper sleeve 7 and a second rotary cap 8. Both ends of the second upper sleeve 7 are connected to the lower sleeve 3 and the second rotary cap 8, respectively. The second rotary cap 8 is provided with a snap-on lid 81. A side, close to the lower sleeve 3, of the second upper sleeve 7 is provided with a second outer wall 701 and a second inner wall 702. The second outer wall 701 is longer than the second inner wall 702. In addition, the second outer wall 701 is threadedly connected to the lower sleeve 3, and the second inner wall 702 is disposed in the lower sleeve 3 and abuts against the guide cylinder 2. A protruding disc 71 is connected inside the second inner wall 702, a cavity opening 72 is formed on the second inner wall 702, and the bottom of the cavity opening 72 extends above the guide cylinder 2. An arc-shaped sliding slot 73 is formed on a side, close to the second rotary cap 8, of the second upper sleeve 7, and the second rotary cap 8 is provided with a sheath rod 82 matched with the arc-shaped sliding slot 73. A top end of the second rotary cap 8 abuts against the protruding disc 71, such that the liquid storage chamber 4 is formed between the interior of the second rotary cap 8 and the protruding disc 71. When the second rotary cap 8 is rotated to be separated from the protruding disc 71, the lubricant enters the guide cylinder 2 via the cavity opening 72, wets and lubricates the catheter 1. An end, close to the lower sleeve 3, of the second upper sleeve 7 is provided with a second tamper-evident ring 74. The lubricant is contained in the liquid storage chamber 4 formed by assembling the second rotary cap 8 to the first upper sleeve 5. When there is a need to lubricate the catheter 1, the second rotary cap 8 is rotated relative to the first upper sleeve 5, the sheath rod 82 is guided along the arc-shaped sliding slot 73, a lower end of the second rotary cap 8 is separated from the protruding disc 71 of the first upper sleeve 5, and the lubricant flows into the guide cylinder 2 from the cavity opening 72, wets and lubricates the catheter 1.

The method for using the portable intermittent urinary catheter with lubrication includes the following steps:

In S1b, the second rotary cap 8 is rotated, such that the second rotary cap 8 is separated from the protruding disc 71, and the lubricant in the liquid storage chamber 4 enters the guide cylinder 2 via the cavity opening 72, wets and lubricates the catheter 1.

In S2b, the second upper sleeve 7 is rotated, such that the second upper sleeve 7 is separated from the lower sleeve 3, and as the second upper sleeve 7 rotates, the second tamper-evident ring 74 is caused to break.

In S3b, the rotary handle 21 is rotated, such that the catheter 1 undergoes an extension motion along with the sheath shaft 14 relative to the guide cylinder 2, the base 13 is detached from the guard plug 22, and the interior of the catheter 1 is in communication with the drainage ports 20.

In S4b, a head portion of the catheter 1 extends and is inserted into the urethra of the user. Then, as the catheter 1 gradually extends, the catheter 1 is gradually screwed into the urethra of the user bit by bit, and urine enters the interior of the catheter 1 via the urine drainage holes 12 and is drained via the drainage ports 20. The drained urine may be directed directly into a toilet or be connected to a urine collection bag via an intervening connector.

In S5b, upon completion of urine drainage, the rotary handle 21 is rotated in a reverse direction, such that the catheter 1 undergoes a retraction motion along with the sheath shaft 14 relative to the guide cylinder 2, the base 13 of the catheter 1 is embedded with the guard plug 22, the second upper sleeve 7 assembled with the second rotary cap 8 is sealed to the lower sleeve 3 by threaded connection, and by means of the rotation of the second rotary cap 8, the second rotary cap and the second upper sleeve 7 return to their original positions.

The portable intermittent urinary catheter with lubrication has good concealment, and accommodates the catheter 1 therein in a concealed manner. The overall design is compact and is convenient for the user to carry, thereby minimizing the risk of privacy exposure. The urinary catheter can be operated by the user independently, or by other nursing staff.

The lubricant is directly pre-stored in the liquid storage chamber 4 above the catheter 1, without the need for an individually packaged lubricant. Simply by rotating the first rotary cap 6 or the second rotary cap 8, the lubricant can be released, such that the catheter 1 can be lubricated quickly, thereby avoiding the splashing of the lubricant when the packaging of the lubricant is torn open, and also reducing the possibility that the catheter 1 is exposed to the air after the packaging bag is torn open and is contaminated by the outside. During the lubrication of the catheter 1, the user's hand does not come into contact with the catheter 1. The catheter 1 is provided with the telescopic assembly M, and by rotating the rotary handle 21, the extension or retraction of the catheter 1 is enabled. During the insertion of the catheter 1 into the urethra, non-contact insertion is enabled throughout the process, thereby reducing the possibility of urethral infection. In addition, during the insertion of the catheter 1 into the urethra, the catheter is advanced toward the urethra bit by bit; that is, the catheter 1 is screwed into the urethra bit by bit, rather than being fully extended at once, which would expose the entire catheter 1 to the air. In this way, the possibility of infection caused by inserting the entire catheter 1 into the urethra while it is exposed to the air is avoided. After use, the catheter 1 is retracted for accommodation, such that the catheter 1 can be resealed and discarded, thereby preventing the generated waste from causing contamination to the environment.

The foregoing descriptions are merely some embodiments of the present disclosure. It should be noted that those of ordinary skill in the art can make other variations and improvements without departing from the inventive concept of the present disclosure, and these variations and improvements all fall within the protection scope of the present disclosure.

What is claimed is:

1. A portable intermittent urinary catheter with lubrication, comprising a catheter, the catheter is a tube body with a hollow structure, urine drainage holes are formed on a tube wall at a front end of the catheter, wherein the catheter further comprises:
a telescopic assembly, a rear end of the catheter being mounted on the telescopic assembly, the telescopic assembly being configured to drive the catheter to extend out of or retract into the telescopic assembly, the telescopic assembly being provided with drainage ports, wherein when the catheter extends out of the telescopic assembly, urine enters the catheter via the urine drainage holes and is discharged through the drainage ports; and
a liquid storage assembly, the liquid storage assembly being detachably connected to the telescopic assembly and covering the front end of the catheter, the liquid storage assembly being provided with a liquid storage chamber therein, the liquid storage chamber containing a lubricant, wherein when the liquid storage chamber is in communication with the telescopic assembly, the lubricant in the liquid storage chamber wets and lubricates the catheter.

2. The portable intermittent urinary catheter with lubrication according to claim 1, wherein the telescopic assembly comprises a lower sleeve, a guide cylinder, and a sheath shaft; a rotary handle is disposed at a rear end of the guide cylinder, and the guide cylinder and the rotary handle form a step-like structure; the guide cylinder is a hollow tubular body, and a grille comprising a plurality of drainage ports is disposed in the guide cylinder; the lower sleeve is sleeved over the guide cylinder; a helical groove is formed on an inner wall of the lower sleeve; a Z-shaped sliding slot is formed on a cylinder body of the guide cylinder; a base having a stepped boss is disposed at the rear end of the catheter; the base is mounted to the sheath shaft, the sheath shaft is assembled in the Z-shaped sliding slot of the guide cylinder and is fitted into the helical groove of the lower sleeve, and when the rotary handle is rotated, the catheter extends or retracts along with the sheath shaft relative to the guide cylinder.

3. The portable intermittent urinary catheter with lubrication according to claim 2, wherein a guard plug is disposed in a center of the grille, the guard plug protrudes toward the catheter and is embedded in the base of the catheter, and when the catheter extends along with the sheath shaft relative to the guide cylinder, the base is detached from the guard plug.

4. The portable intermittent urinary catheter with lubrication according to claim 3, wherein a hydrophilic coating is applied to a surface of the catheter, and a physiological saline solution is used as the lubricant.

5. The portable intermittent urinary catheter with lubrication according to claim 4, wherein the liquid storage assembly comprises a first upper sleeve and a first rotary cap; both ends of the first upper sleeve are connected to the lower sleeve and the first rotary cap respectively; a sealing sheet is disposed on a side of the first upper sleeve adjacent to the lower sleeve; the first rotary cap, the first upper sleeve, and the sealing sheet form the liquid storage chamber; a cutter is disposed in the first rotary cap; after the cutter breaks the sealing sheet, the lubricant in the liquid storage chamber enters the guide cylinder, wets and lubricates the catheter.

6. The portable intermittent urinary catheter with lubrication according to claim 5, wherein a side of the sealing sheet facing the first rotary cap is provided with a flange perpendicular to the sealing sheet; the cutter comprises a curved surface, a protruding rib, and a tip; the curved surface and the protruding rib are connected to a side of the first rotary cap, a vertical surface of the protruding rib is connected to the curved surface, and the tip is disposed at an end of the curved surface; when the first rotary cap is rotated, the tip presses against and cuts the flange of the sealing sheet to break the sealing sheet.

7. The portable intermittent urinary catheter with lubrication according to claim 6, wherein a side of the first upper sleeve close to the lower sleeve is provided with a first outer wall and a first inner wall; the first outer wall is longer than the first inner wall, and the first outer wall is threadedly connected to the lower sleeve, and the first inner wall is disposed in the lower sleeve and abuts against the guide cylinder; the sealing sheet is disposed on the first inner wall of the first upper sleeve; a sealing ring is disposed at a junction of the first rotary cap and the first upper sleeve.

8. The portable intermittent urinary catheter with lubrication according to claim 7, wherein a first tamper-evident ring is disposed at an end of the first upper sleeve adjacent to the lower sleeve; a plurality of clips are disposed in the first tamper-evident ring; when the first upper sleeve is unscrewed, the clips are pressed by threads, such that the first tamper-evident ring is broken.

9. The portable intermittent urinary catheter with lubrication according to claim 4, wherein the liquid storage assembly comprises a second upper sleeve and a second rotary cap, and both ends of the second upper sleeve are connected to the lower sleeve and the second rotary cap respectively; a side of the second upper sleeve close to the lower sleeve is provided with a second outer wall and a second inner wall; the second outer wall is longer than the second inner wall, and the second outer wall is threadedly connected to the lower sleeve, and the second inner wall is disposed in the lower sleeve and abuts against the guide cylinder; a protruding disc is connected to the second inner wall, a cavity opening is formed on the second inner wall, and a bottom of the cavity opening extends above the guide cylinder; an arc-shaped sliding slot is formed on a side of the second upper sleeve adjacent to the second rotary cap, and the second rotary cap is provided with a sheath rod matched with the arc-shaped sliding slot; a top end of the second rotary cap abuts against the protruding disc, such that the liquid storage chamber is formed between the second rotary cap and the protruding disc; when the second rotary cap is rotated to be separated from the protruding disc, the lubricant enters the guide cylinder via the cavity opening, wets and lubricates the catheter; an end of the second upper sleeve adjacent to the lower sleeve is provided with a second tamper-evident ring.

10. A method for using the portable intermittent urinary catheter with lubrication according to claim 8, comprising steps of:
S1a, rotating the first rotary cap, such that the first rotary cap is misaligned with the first upper sleeve, and the tip of the first rotary cap presses and cuts the flange of the sealing sheet to break the sealing sheet, and the lubricant in the liquid storage chamber enters the guide cylinder, wets and lubricates the catheter; and rotating the first upper sleeve, the clips of the first tamper-evident ring are pressed by threads, such that the first tamper-evident ring is broken;
S2a, continuing to rotate the first upper sleeve, such that the first upper sleeve is separated from the lower sleeve;
S3a, rotating the rotary handle, such that the catheter extends along with the sheath shaft relative to the guide cylinder, the base is detached from the guard plug, and an interior of the catheter is in communication with the drainage ports;
S4a, allowing the catheter to gradually extend and be screwed into the urethra of a user, such that urine enters the interior of the catheter via the urine drainage holes and is discharged through the drainage ports, and the drained urine may be directly directed into a toilet or into a urine collection bag via an intervening connector; and
S5a, upon completion of urine drainage, rotating the rotary handle in a reverse direction, such that the catheter retracts along with the sheath shaft relative to the guide cylinder, the guard plug is embedded in the base of the catheter, and the first upper sleeve assembled with the first rotary cap is sealed to the lower sleeve by threaded connection.

11. A method for using the portable intermittent urinary catheter with lubrication according to claim 9, comprising steps of:
S1b, rotating the second rotary cap, such that the second rotary cap is separated from the protruding disc, and the lubricant in the liquid storage chamber enters the guide cylinder via the cavity opening, wets and lubricates the catheter;
S2b, rotating the second upper sleeve, such that the second upper sleeve is separated from the lower sleeve, and as the second upper sleeve rotates, the second tamper-evident ring is broken;
S3b, rotating the rotary handle, such that the catheter extends along with the sheath shaft relative to the guide cylinder, the base is detached from the guard plug, and an interior of the catheter is in communication with the drainage ports;
S4b, allowing the catheter to gradually extend and be screwed into the urethra of the user, such that urine enters the interior of the catheter via the urine drainage holes and is discharged through the drainage ports, and the drained urine may be directly directed into a toilet or into a urine collection bag via an intervening connector; and
S5b, upon completion of urine drainage, rotating the rotary handle in a reverse direction, such that the catheter retracts along with the sheath shaft relative to the guide cylinder, the guard plug is embedded in the base of the catheter, the second upper sleeve assembled with the second rotary cap is sealed to the lower sleeve by threaded connection, and the second rotary cap is rotated back to be reset relative to the second upper sleeve.
